(19) United States
(12) Patent Application Publication
(10) Pub. No.: US 2026/0202070 A1
(43) Pub. Date: Jul. 16, 2026

(54) AIR PURIFIER AND AIR-CONDITIONING APPARATUS

(71) Applicant: Mitsubishi Electric Corporation, Tokyo (JP)

(72) Inventors: Akinori SHIMIZU, Tokyo (JP); Akane NOMURA, Tokyo (JP)

(73) Assignee: Mitsubishi Electric Corporation, Tokyo (JP)

(21) Appl. No.: 19/127,759

(22) PCT Filed: Nov. 22, 2022
(86) PCT No.: PCT/JP2022/043176

§ 371 Date: May 7, 2025

(51) Int. Cl. F24F 8/30; A61L 9/22

(52) U.S. Cl. CPC F24F 8/30; A61L 9/22; A61L 2209/16

(57) ABSTRACT

An air purifier includes a plurality of first discharge electrodes, and is configured to supply a discharge product generated by the plurality of first discharge electrodes toward a treatment target within a space to purify the air in the space, the plurality of first discharge electrodes are formed to extend in a first direction, and being electrodes of a same polarity arranged spaced apart in a second direction orthogonal to the first direction, and the air purifier includes an induction electrode that is configured to form an electric field between the induction electrode and the plurality of first discharge electrodes, and arranged at a central part of the plurality of first discharge electrodes as viewed in the first direction.

### TECHNICAL FIELD

The present disclosure relates to an air purifier and an air-conditioning apparatus including a generating unit configured to generate chemical species such as discharge product by discharging at a high voltage.

### BACKGROUND ART

There is known an air purifier that sterilizes bacteria or inactivates viruses by supplying, into the air, discharge products generated by applying a high voltage between electrodes and conveying the discharge products to a treatment target such as bacteria or viruses. An air purifier of this kind includes a generator configured to generate discharge products such as ions. One such conventional generator is an ion generating apparatus of Patent Literature 1. The ion generating apparatus of Patent Literature 1 includes a plurality of bar-shaped discharge electrodes arranged in parallel with each other, a plurality of induction electrodes arranged to face the plurality of discharge electrode in an axial direction of the discharge electrodes, and a high voltage application unit configured to apply a high voltage between the discharge electrode and the induction electrode. The ion generating apparatus of Patent Literature 1 is configured to generate a discharge product between the plurality of discharge electrodes and the plurality of counter electrodes.

### CITATION LIST

### Patent Literature

Patent Literature 1: JP2012-79423 A

### SUMMARY OF INVENTION

### Technical Problem

The ion generating apparatus disclosed in Patent Literature 1 has multiple discharge electrodes charged to a same polarity, and most parts of the discharge product generated from these discharge electrodes are also charged to a same polarity. Since molecules of at least one discharge product charged to the same polarity repel each other, there is a risk that the molecules of the at least one discharge product may diffuse due to this repulsion, making it difficult to establish a stable supply direction. Therefore, when the ion generating apparatus disclosed in Patent Literature 1 is applied to an air purifier, the at least one discharge product is not conveyed to the treatment target at the concentration necessary for sterilizing bacteria or inactivating viruses. As a result, the air purifier is unable to sufficiently achieve the effects of bacterial sterilization and virus inactivation (hereinafter referred to as “sterilization and virus inactivation effects”), problematically.

The present disclosure has been made in view of the foregoing issues, and its objective is to provide an air purifier and an air-conditioning apparatus capable of suppressing the diffusion of a discharge product and enhancing the sterilization and virus inactivation effects

### Solution to Problem

An air purifier of an embodiment of the present disclosure includes a plurality of first discharge electrodes and is configured to supply a discharge product generated by the plurality of first discharge electrodes toward a treatment target within a space to purify the air in the space, the plurality of first discharge electrodes being formed to extend in a first direction, and being electrodes of a same polarity arranged spaced apart in a second direction orthogonal to the first direction, the air purifier including an induction electrode configured to form an electric field between the induction electrode and the plurality of first discharge electrodes, and arranged at a central part of the plurality of first discharge electrodes as viewed in the first direction.

An air-conditioning apparatus of an embodiment of the present disclosure includes an air purifier of the above; a heat exchanger configured to cause refrigerant flowing inside and air present around the heat exchanger to exchange heat with each other; and an air blowing unit arranged upstream of the plurality of first discharge electrodes and configured to generate airflow, and supply the discharge product into the space, wherein the air-conditioning apparatus is configured such that air supplied by the air blowing unit passes through the heat exchanger, and the air that is air-conditioned by passing through the heat exchanger supplies the discharge product into the space.

### Advantageous Effects of Invention

The air purifier and the air-conditioning apparatus of embodiments of the present disclosure include a plurality of discharge electrodes formed to extend in a first direction, and being electrodes of a same polarity arranged spaced apart in a second direction orthogonal to the first direction. The air purifier includes an induction electrode configured to form an electric field between the induction electrode and the plurality of first discharge electrodes, and arrange at a central part of the plurality of first discharge electrodes as viewed in the first direction. Therefore, the air purifier can collect discharge products generated from the plurality of discharge electrodes towards the induction electrode such that the discharge products converge in a direction that brings them closer together when viewed in the first direction, so that diffusing of the discharge product can be suppressed and the concentration is thus increased. Therefore, the air purifier is capable of transporting the discharge products in a highly concentrated state toward the treatment target and enhancing the sterilization and virus inactivation effects.

### BRIEF DESCRIPTION OF DRAWINGS

FIG. 1 is a schematic perspective view of an air purifier according to Embodiment 1.

FIG. 2 is a schematic perspective view of a generating unit of the air purifier according to Embodiment 1, viewed from below.

FIG. 3 is a schematic side-view of the generating unit of an air purifier according to Embodiment 1.

FIG. 4 shows an electric field formed around a plurality of first discharge electrodes in a comparative example.

FIG. 5 is a schematic diagram showing a behavior of a discharge product in the comparative example.

FIG. 6 shows an electric field formed between the induction electrode and the first discharge electrode in the air purifier according to Embodiment 1.

FIG. 7 is a schematic diagram showing the behavior of a discharge product of air purifier according to Embodiment 1.

FIG. 8 is a perspective view showing a modification of an air purifier according to Embodiment 1.

FIG. 9 is a schematic side-view of an air purifier according to Embodiment 2.

FIG. 10 is a schematic diagram showing behavior of a discharge product the air purifier according to Embodiment 2.

FIG. 11 is a schematic diagram showing behavior of a discharge product of an air purifier according to Embodiment 1, as a comparative example.

FIG. 12 is a schematic perspective view of an air purifier according to Embodiment 3.

FIG. 13 is a schematic perspective view of a generating unit of the air purifier according to Embodiment 3, viewed from below.

FIG. 14 is a schematic cross-section of a generating unit of an air purifier according to Embodiment 3.

FIG. 15 is a schematic diagram showing behavior of the discharge product of the air purifier according to Embodiment 3.

FIG. 16 is a schematic perspective view of an air purifier according to Embodiment 4.

FIG. 17 is schematic perspective view of a generating unit of the air purifier according to Embodiment 4, viewed from below.

FIG. 18 is a schematic cross-section of the generating unit of an air purifier according to Embodiment 4.

FIG. 19 is a schematic diagram showing one example of an air-conditioning apparatus according to Embodiment 5.

### DESCRIPTION OF EMBODIMENTS

Hereinafter, the air purifier according to the embodiment will be described with reference to the drawings and other materials. In the drawings explained below, components indicated by the same reference numerals are the same as or equivalent to each other, and this applies throughout the entire description of the embodiments. Additionally, the size relationships of the individual components in the drawings may differ from those in actual configurations. The forms of the components represented in the entirety of this specification are merely exemplary and are not limited to the forms described herein. In particular, the combinations of components are not limited to the combinations described in each embodiment, and components described in one embodiment may be applied to another embodiment

### Embodiment 1

[General Configuration of Air Purifier 1]

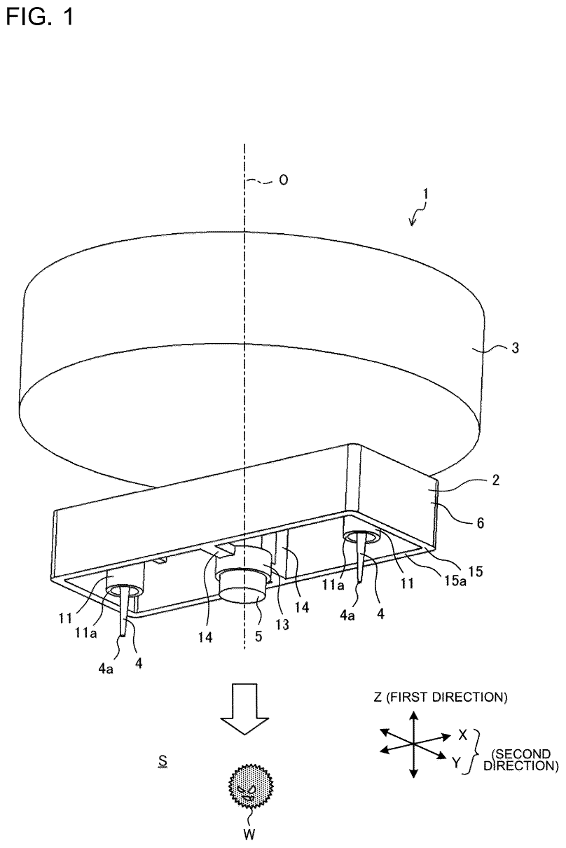

FIG. 1 is a schematic perspective view of an air purifier according to Embodiment 1.

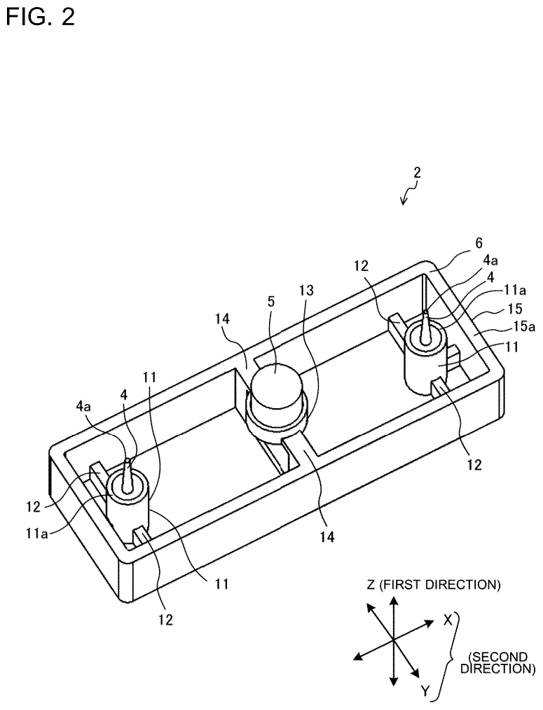

FIG. 2 is a schematic perspective view of the generating unit of the air purifier according to Embodiment 1, viewed from below.

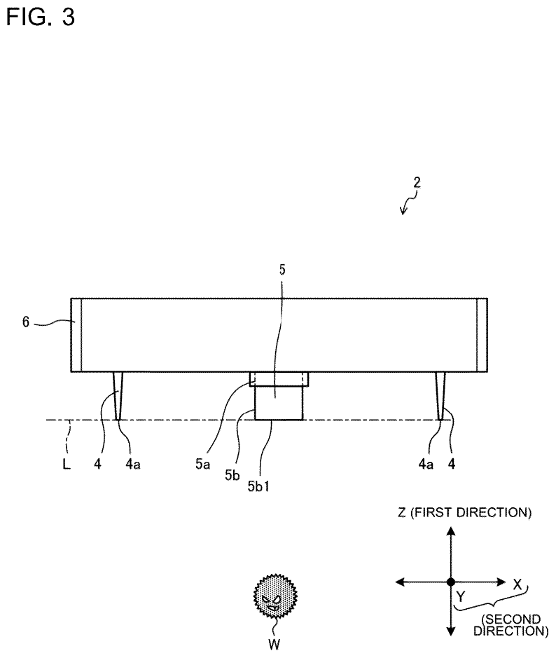

FIG. 3 is a schematic side-view of the generating unit 2 of the air purifier 1 according to Embodiment 1.

In the drawings, three arrows X, Y, and Z show directions orthogonal to each other. X direction translates into a right-left direction, Z direction translates into upward and downward direction, and Y direction translates into forward-back direction.

The air purifier 1 is a device that purifies the air in space S where the air purifier 1 is installed. More specifically, the air purifier 1 is a device that sterilizes bacteria or inactivates viruses present in the space. The air purifier 1 may also include configurations as part of an air-conditioning apparatus with functions such as, for example, temperature regulation and moisture control, or as a device with ventilation functionality.

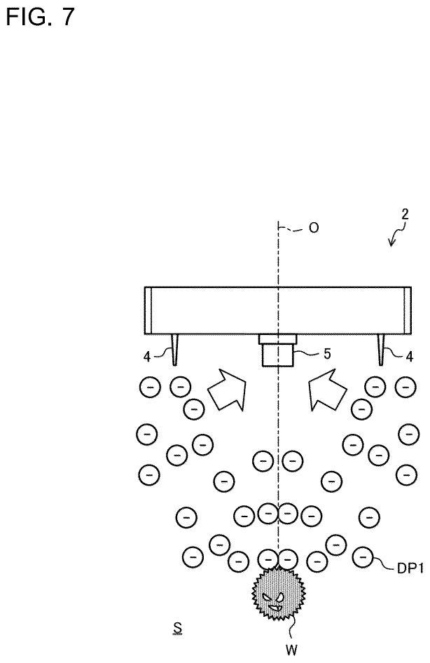

The air purifier 1 includes a high voltage converter (not shown) that transforms the input electric voltage into a high voltage, the generating unit 2 that generates a discharge product DP1 (see FIG. 7 described below), and an air blowing unit 3 that supplies the discharge product DP1 generated by the generating unit 2 into the space S. The discharge product DP1 is supplied into the space S in the direction indicated by the hollow arrow in FIG. 1. The discharge product DP1 supplied into the space S is conveyed to the treatment target W present in the space S and processes the treatment target W. The treatment target W may be bacteria, viruses, or both. “Processes the treatment target W” refers to sterilizing bacteria, inactivating viruses, or both.

### (Generating Unit 2)

The generating unit 2 includes a plurality of first discharge electrodes 4, an induction electrode 5, and a frame-shaped retaining member 6 that holds the plurality of first discharge electrodes 4 and the induction electrode 5. The first discharge electrodes 4 are formed to extend in a first direction, as indicated by the Z direction in the drawing. The plurality of first discharge electrodes 4 are arranged spaced apart in a second direction orthogonal to the first direction. The second direction is a planar direction orthogonal to the first direction and includes the X and Y directions. Here, the first discharge electrodes 4 comprise two electrodes; however, the number is not limited to two, and any number, greater than or equal to two, of the electrodes may be provided.

The first discharge electrode 4 is composed of a conical needle-shaped electrode that tapers in diameter from the base end to the tip end. In the first discharge electrode 4, one end in the first direction is the tip end, while the other end is the base end. The first discharge electrode 4 is retained at its base by the first retaining unit 11, which is part of the retaining member 6, as described later. The first discharge electrode 4 has a discharge point 4a at its tip end, formed by a needle-like tip where discharge occurs. The first discharge electrode 4 is retained by the first retaining unit 11 in such a manner that the discharge point 4a projects below the bottom surface 11a of the first retaining unit 11 and the bottom surface 15a of the frame portion 15, which will be described later. The material of the first discharge electrode 4 is metal. However, the material of the induction electrode is not limited to metal and may be made of other conductive materials, such as carbon fibers with conductivity or similar conductive substances.

The induction electrode 5, similar to the first discharge electrode 4, is formed to extend in the first direction. The induction electrode 5 is cylindrical in shape. The induction electrode 5 is arranged at the central part of the plurality of first discharge electrodes 4 as viewed in the first direction. The induction electrode 5 is aligned in parallel with the plurality of first discharge electrodes 4. The material of the induction electrode 5 is metal. However, the material of the induction electrode 5 is not limited to metal and may also be any of other conductive materials, such as carbon fibers with conductivity.

As shown in FIG. 3, the induction electrode 5 includes a base end 5a, which is retained by the second retaining unit 13 of the retaining member 6, which will be described later, and a tip end 5b, located on the side opposite to the base end 5a. The induction electrode 5 is arranged such that a tip-end surface 5b1 of the tip end 5b overlaps with a virtual line L that connects the discharge points 4a of the plurality of first discharge electrodes 4.

The induction electrode 5 is arranged such that, in terms of its position in the second direction, a central axis O of the induction electrode 5 aligns with the central axis of the air blowing unit 3. It should be noted that the alignment of the central axis O of the induction electrode 5 with the central axis of the casing of the air purifier 1 (not shown) is not specifically restricted; the central axes may or may not align.

The plurality of first discharge electrodes 4 and the induction electrode 5 are retained by the retaining member 6. The plurality of first discharge electrodes 4 and the induction electrode 5 are retained at the same ends in the first direction by the retaining member 6. The retaining member 6 includes the first retaining unit 11 that retains the first discharge electrodes 4, a second retaining unit 13, which holds the induction electrode 5, and a rectangular frame portion 15. The first retaining unit 11, the second retaining unit 13, and the frame portion 15 are integrally formed. The material of the retaining member 6 is resin. However, the material of the retaining member 6 is not limited to resin and may be made of other materials with high electrical insulation, such as ceramic with excellent electrical insulation property.

The first retaining unit 11 is provided in the same number as the first discharge electrodes 4, which in this case is two. The two first retaining units 11 are spaced apart from each other and fixed inside the frame portion 15. The first retaining unit 11 is formed in a cylindrical shape to surround the first discharge electrode 4. The first retaining unit 11 is fixed to the frame portion 15 by a pair of fixing legs 12 that extend in opposite directions from the outer circumferential surface of the first retaining unit 11. The first discharge electrode 4 is fixed to the first retaining unit 11 by having the end opposite to the discharge point 4a inserted into the first retaining unit 11. The first discharge electrode 4 is fixed to the first retaining unit 11 by having a fixing member (not shown) inserted between the outer circumferential surface of the first discharge electrode 4 and the inner circumferential surface of the first retaining unit 11.

The second retaining unit 13 is positioned at the center of the two second retaining units 13 as viewed in the first direction. The second retaining unit 13 is formed in a cylindrical shape that surrounds the induction electrode 5. The second retaining unit 13 is fixed to the frame portion 15 by the pair of fixing legs 14 that extend in opposite directions from the outer circumferential surface of the second retaining unit 13. The induction electrode 5 is retained by the second retaining unit 13 with its base end 5a inserted into the second retaining unit 13. The induction electrode 5 is fixed within the second retaining unit 13 by having a fixing member (not shown) inserted between the outer circumferential surface of the induction electrode 5 and the inner circumferential surface of the second retaining unit 13. The fixing member can be, for example, an electrically insulating resin part, potting material, or adhesive.

The first discharge electrode 4 ionizes the air in its proximity by being applied with the high voltage obtained from the high voltage converter, thereby generating discharge products DP1, such as negative ions or positive ions. Electricity of a same polarity is applied to the plurality of first discharge electrodes 4. In other words, the plurality of first discharge electrodes 4 are electrodes of the same polarity. Thus, all of the plurality of first discharge electrodes 4 have the same polarity. The polarity of electricity applied to the first discharge electrode 4 is negative in this case; however, it is not limited to negative polarity and may also be positive polarity.

The polarity of the ions generated from the first discharge electrode 4 depends on the polarity of the high voltage applied by the high voltage converter. When the polarity of the high voltage applied by the high voltage converter is negative, the ion generated from the first discharge electrode 4 is a negative ion, and when the polarity is positive, the ion is a positive ion. When a high voltage is applied to the negative pole of the first discharge electrode 4, electrons are emitted from the first discharge electrode 4. These electrons combine with oxygen or water molecules present in the air near the first discharge electrode 4, resulting in the generation of the discharge product DP1.

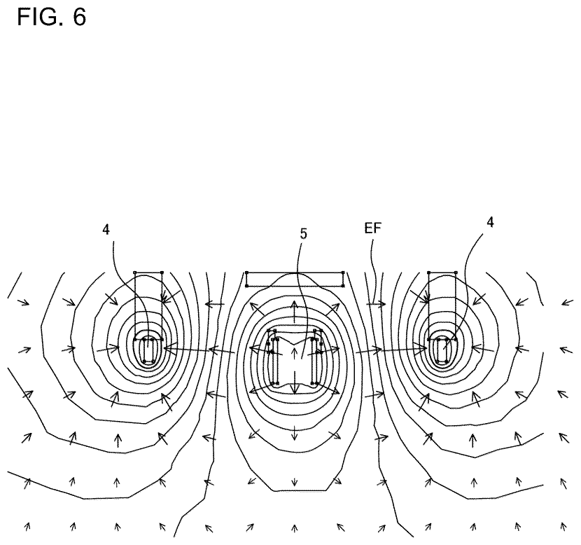

The first discharge electrode 4, being a needle-shaped electrode, allows the electric field formed between the first discharge electrode 4 and the induction electrode 5 to concentrate at the discharge point 4a at the tip-end of the first discharge electrode 4. When a negative high voltage is applied to the first discharge electrode 4, the emission of electrons from the first discharge electrode 4 is easier when the electric field EF (see FIG. 6 described below) formed between the first discharge electrode 4 and the induction electrode 5 is concentrated on the first discharge electrode 4 compared to when it is not concentrated. Since the first discharge electrode 4 is a needle-shaped electrode, as described earlier, it enables the concentration of the electric field at the discharge point 4a at the tip of the first discharge electrode 4, facilitating the emission of electrons. It should be noted that, for the above reasons, a shape with a tapered tip, such as a needle-shaped electrode, is desirable for the first discharge electrode 4. However, this shape is not limited to a needle shape with a tapered end and may be columnar. Additionally, the first discharge electrode 4 may also take the form of an electrode made of thin wires or a brush-shaped electrode composed of a bundle of multiple thin wires. Additionally, the first discharge electrode 4 may also take the form of an electrode made of thin wires or a brush-shaped electrode composed of a bundle of multiple thin wires.

The induction electrode 5 forms, with the plurality of first discharge electrodes 4, an electric field EF therebetween and attracts the discharge product DP1 generated from the first discharge electrode 4. The induction electrode 5 is either grounded or has a polarity different from that of the first discharge electrode 4. By applying a voltage to the first discharge electrodes 4, an electric field EF is formed between the induction electrode 5 and the first discharge electrodes 4

### (Air Blowing Unit 3)

The air blowing unit 3 is configured as a fan, forming an airflow and generating wind. The air blowing unit 3 supplies the discharge product DP1 generated by the generating unit 2 into the space S via the airflow. The discharge product DP1 supplied into the space S reaches surfaces of furniture such as desks and other interior objects, acting on and treating bacteria and viruses adhered to those surfaces. The air blowing unit 3 is arranged near the generating unit 2. The air blowing unit 3 is positioned upstream of the generating unit 2. If the air blowing unit 3 are placed downstream of the generating unit 2, the discharge product DP1 generated by the generating unit 2 might be stirred and diffused while passing through the air blowing unit 3. For this reason, the air blowing unit 3 is arranged upstream of the generating unit 2.

The fan is, for example, an axial flow propeller fan. By adopting an axial flow propeller fan as the air blowing fan, the air blowing fan can generate a large-volume airflow. The motor connected to the air blowing fan is typically a standard AC capacitor motor, but it is not limited to AC capacitor motors.

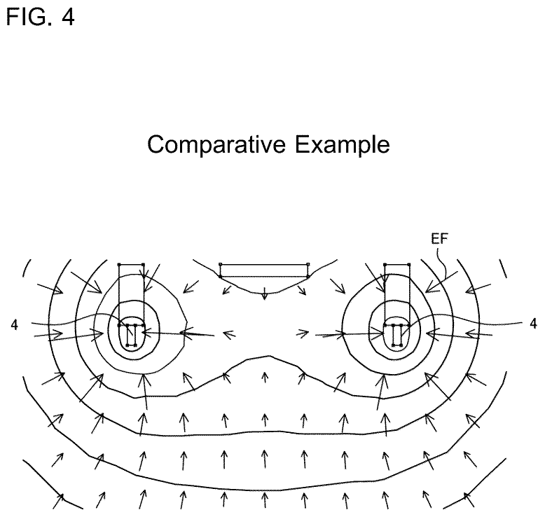

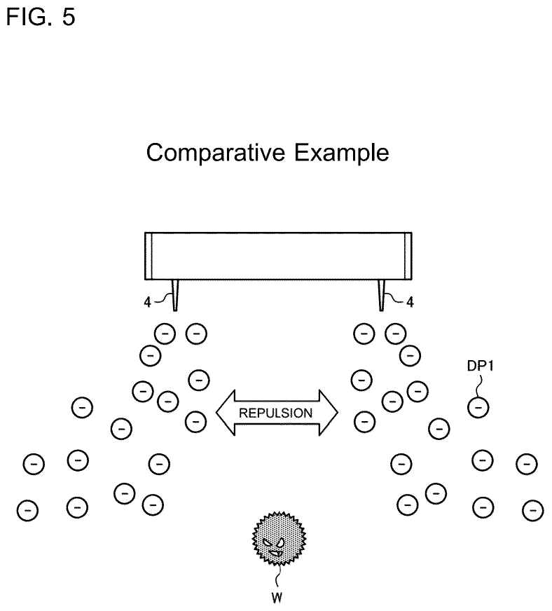

Next, the operation of the above-described configuration will be explained. First, as a comparative example, a configuration without an induction electrode 5 between the plurality of first discharge electrodes 4 will be described with reference to FIG. 4 and FIG. 5.

FIG. 4 shows the electric field EF formed around the plurality of first discharge electrodes 4 in a comparative example. FIG. 5 is a schematic diagram showing the behavior of the discharge product DP1 in the comparative example. In FIG. 4, there is no induction electrode 5 between the plurality of first discharge electrodes 4, and it shows the case where the high voltage applied to the first discharge electrode 4 is of negative polarity. Because the high voltage applied to the first discharge electrode 4 is of negative polarity, the direction of the electric field EF is oriented from the outside toward the first discharge electrode 4. In FIG. 4, the electric field EF is indicated by arrows. Since the high voltage applied to the first discharge electrode 4 is of negative polarity, as shown in FIG. 5, negative ions are generated from the first discharge electrode 4 as the discharge product DP1. Negative ions carry a negative charge, causing them to experience a force in the direction opposite to the electric field EF. As a result, in the comparative example, as shown in FIG. 5, negative ions generated from the plurality of first discharge electrodes 4 of the same polarity repel each other and diffuse. Consequently, in the comparative example, the concentration of the discharge product DP1 reaching the treatment target W is reduced.

FIG. 6 shows the electric field EF formed between the first discharge electrode 4 and the induction electrode 5 in the air purifier 1 according to Embodiment 1. FIG. 7 is a schematic diagram showing the behavior of the discharge product DP1 in the air purifier 1 according to Embodiment 1. Both FIG. 6 and FIG. 7 depict the case where the high voltage applied to the first discharge electrode 4 is of negative polarity.

Since the high voltage applied to the first discharge electrode 4 is of negative polarity, the direction of the electric field EF is oriented from the induction electrode 5 toward the first discharge electrode 4. Additionally, because the high voltage applied to the first discharge electrode 4 is of negative polarity, negative ions are generated from the first discharge electrode 4. Negative ions carry a negative charge, and thus experience a force in the direction opposite to the electric field EF. Therefore, as shown in FIG. 7, the negative ions are subjected to a force that drives them from the first discharge electrode 4 toward the induction electrode 5. As indicated by the hollow arrows in FIG. 7, the negative ions are attracted to the induction electrode 5.

Here, if the electric field EF is E [V/m] and the electric charge of the discharge product DP1 is q [C], the force F [N] acting on the discharge product DP1 can be expressed by the following equation:

F=qE

As described above, the induction electrode 5 is positioned at the central part of the plurality of first discharge electrodes 4 when viewed in the first direction. Therefore, the negative ions generated from each of the plurality of first discharge electrodes 4 are gathered at the center of the plurality of first discharge electrodes 4 when viewed in the first direction. In other words, the negative ions generated from each of the plurality of first discharge electrodes 4 are directed toward the induction electrode 5, converging in a direction that brings them closer together when viewed in the first direction. Additionally, the induction electrode 5 is arranged in parallel with the plurality of first discharge electrodes 4. If the induction electrode 5 is not arranged in parallel with the plurality of first discharge electrodes 4, meaning it is positioned to face the plurality of first discharge electrodes 4 in the first direction, there could be a possibility of repulsion among the discharge products DP1, as shown in FIG. 5. For this reason, the induction electrode 5 is arranged in parallel with the plurality of first discharge electrodes 4.

In this manner, the air purifier 1 can collect the negative ions generated from each of the plurality of first discharge electrodes 4 in a direction that brings them closer together, suppressing the diffusion of negative ions and increasing their concentration Therefore, even if the treatment target W is located at a distance from the air purifier 1, the air purifier 1 can transport the negative ions to the treatment target W while maintaining a high concentration. Thus, the air purifier 1 can deliver negative ions to the treatment target W at a high concentration, enabling it to achieve a high sterilization and virus inactivation effect.

When the high voltage applied to the first discharge electrode 4 is of positive polarity, the direction of the electric field EF is from the first discharge electrode 4 toward the induction electrode 5. In the case of electricity of positive polarity being applied, the first discharge electrode 4 generates positive ions. Since positive ions carry a positive charge, they experience a force in the same direction as the electric field EF. In other words, positive ions are subjected to a force in the same direction as in the case of negative ions, moving from the first discharge electrode 4 toward the induction electrode 5. The positive ions are thus attracted to the induction electrode 5. Therefore, the air purifier 1 can achieve the same working and effects as described above, even when the high voltage applied to the first discharge electrode 4 is of positive polarity, as in the case of negative polarity.

In FIG. 1, the first discharge electrodes 4 are two in number; however, the first discharge electrodes 4 may be any number as long as there are two or more

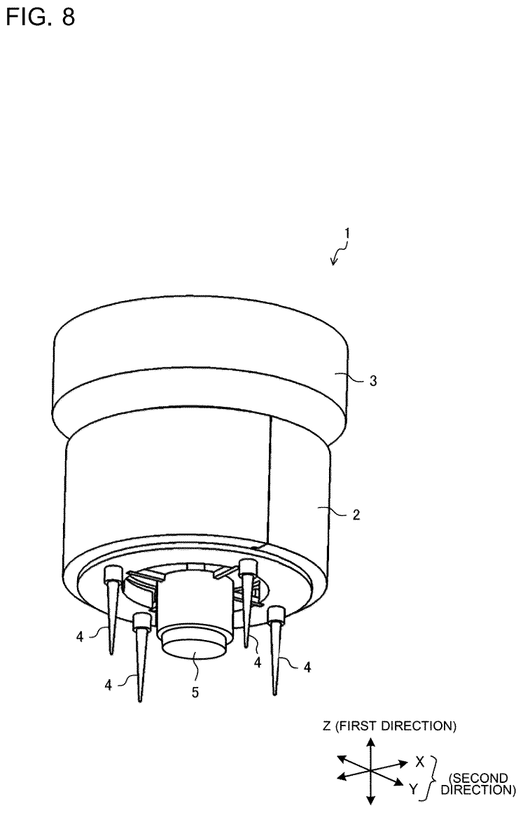

The following FIG. 8 illustrates an example of an arrangement where there are four first discharge electrodes 4.

FIG. 8 is a perspective view showing a modification of the air purifier 1 according to Embodiment 1. In the modification shown in FIG. 4, there are four first discharge electrodes 4, which are arranged in a circular configuration spaced apart in the second direction. Additionally, the induction electrode 5 is positioned at the center of the four first discharge electrodes 4 when viewed in the first direction.

Even with the above-described configuration, the same working and effects as in the case where there are two first discharge electrodes 4, can be achieved.

### [Effects of Air Purifier 1]

The air purifier 1 includes the plurality of first discharge electrodes 4 and purifies the air in the space S by supplying the discharge product DP1, generated from the plurality of first discharge electrodes 4, toward the treatment target W in the space. The plurality of first discharge electrodes 4 are formed to extend in the first direction and are arranged as electrodes of the same polarity, spaced apart in a second direction orthogonal to the first direction. The air purifier 1A includes the induction electrode 5, which is positioned at the center of the plurality of first discharge electrodes 4 when viewed in the first direction, and forms an electric field with the plurality of first discharge electrodes 4.

With the above-described configuration, the air purifier 1 can collect the discharge product DP1 generated from the plurality of first discharge electrodes 4 toward the induction electrode in a direction that brings them closer together when viewed in the first direction, suppressing diffusion and increasing the concentration of the discharge product. Thus, the air purifier can transport the discharge product DP1 in a highly concentrated state to the treatment target, thereby enhancing its sterilization and virus inactivation effects.

The air purifier 1 includes the retaining member 6 with electrical insulation properties, which holds the plurality of first discharge electrodes 4 and the induction electrode 5. Each of the plurality of first discharge electrodes 4 has a discharge point 4a at one end in the first direction, formed as a needle-shaped tip where discharge occurs. The opposite end of the discharge point 4a is retained by the retaining member 6.

If the tip of the first discharge electrode 4 is not needle-shaped, it becomes necessary to either increase the voltage or position the induction electrode 5 near the first discharge electrode 4 to induce discharge. Increasing the voltage has the disadvantage of requiring a larger and more expensive power supply. Positioning the induction electrode 5 near the first discharge electrode 4 may result in adverse effects, such as the generation of ozone in addition to ions. Moreover, if the end of the first discharge electrode 4 opposite to the discharge point 4a is not retained by the retaining member 6, there is a risk of abnormal discharge or electrical leakage. This could occur if the first discharge electrode 4 comes into contact with the metal casing, which constitutes the outer structure of the air purifier 1, when the voltage is applied.

On the other hand, the air purifier 1 avoids the above-mentioned problems because the tip of the first discharge electrode 4 is needle-shaped, and the end opposite to the discharge point 4a of the first discharge electrode 4 is retained by the retaining member 6.

### Embodiment 2

An air purifier 1A according to Embodiment 2 differs from the air purifier 1 in Embodiment 1 in the position of the tip-end surface 5b1 of the induction electrode 5 in the first direction. Hereafter, the explanation will focus on the differences between Embodiment 2 and Embodiment 1. Configurations not described in Embodiment 2 are the same as those in Embodiment 1.

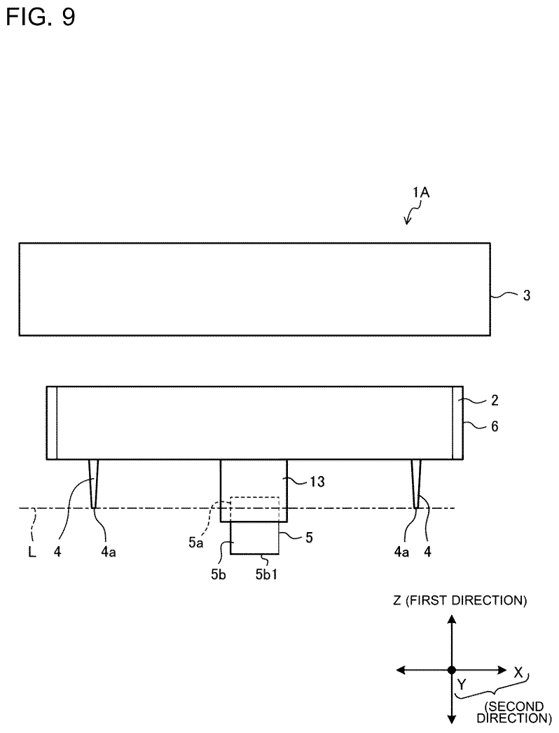

FIG. 9 is a schematic side-view of the air purifier 1A according to Embodiment 2. The air purifier 1A differs from the air purifier 1 in Embodiment 1 in that the tip-end surface 5b1 of the induction electrode 5 is positioned on the treatment target W side from the virtual line L in the first direction. Other configurations are the same as those of the air purifier 1 in Embodiment 1. The specific structure for positioning the tip-end surface 5b1 of the induction electrode 5 as described above is not particularly limited and may be achieved as follows. For example, the air purifier 1A can be configured such that the length of the second retaining unit 13 in the first direction is extended toward the treatment target W compared to the air purifier 1, and the base end 5a of the induction electrode 5 is inserted and fixed inside the extended portion.

The working and effects of the above-described configuration will be explained by comparing the behavior of the discharge product DP1 in Embodiment 2 with that in Embodiment 1.

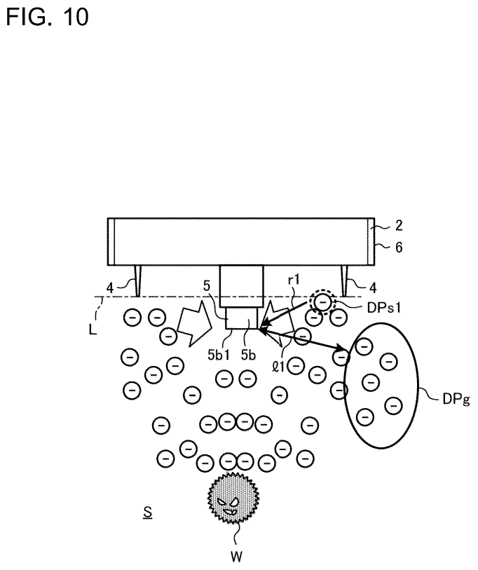

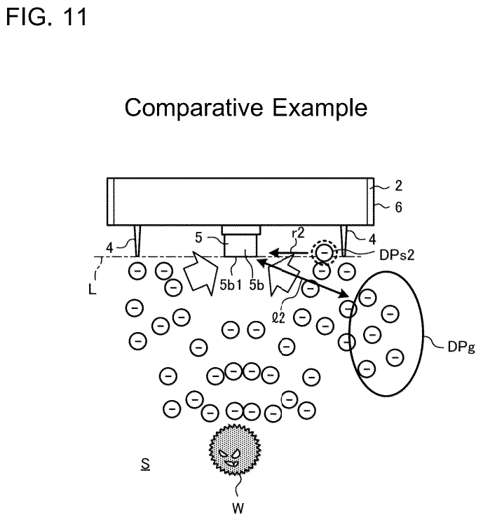

FIG. 10 is a schematic diagram illustrating the behavior of the discharge product DP1 in the air purifier 1A according to Embodiment 2. FIG. 11 is a schematic diagram illustrating the behavior of the discharge product DP1 in the air purifier 1 according to Embodiment 1, shown here as a comparative example. In Embodiment 2, as shown in FIG. 10, the distance between the discharge product group DPg (enclosed by a solid circle) and the induction electrode 5 is closer compared to that in Embodiment 1, as shown in FIG. 11. Specifically, the distance 11 in FIG. 10 is shorter than the distance 12 in FIG. 11. As a result, the air purifier 1A in Embodiment 2 allows the discharge product group DPg to be more effectively subjected to the induction effect of the induction electrode 5, further suppressing the diffusion of the discharge product DP1 and increasing its concentration.

In FIG. 10, the discharge product DPs1, enclosed by a dashed circle, and the discharge product DPs2, similarly enclosed in FIG. 11 by a dashed circle, are discharge products DP1 located at the same distance from the retaining member 6 in the first direction. Because the tip-end surface 5b1 of the induction electrode 5 is positioned on the treatment target W side from the virtual line L, the discharge product DPs1 and the discharge product DPs2 exhibit the following behaviors different from the discharge product DPs2. In FIG. 11, the discharge product DPs2 is attracted laterally toward the induction electrode 5, as indicated by arrow r2. In contrast, in FIG. 10, the discharge product DPs1 is attracted diagonally downward, as shown by arrow r1—that is, in a direction to the treatment target W. As a result, the air purifier 1A can accelerate and attract the discharge product DPs1 toward the treatment target W, increasing the concentration of the discharge product DP1 reaching the treatment target W compared to the air purifier 1.

### [Effects of Air Purifier 1A]

The air purifier 1A achieves the same effects as the air purifier 1, along with the following additional benefits. The air purifier 1A positions the tip-end surface 5b1 of the induction electrode 5 on the treatment target W side from the virtual line L in the first direction. This positioning allows the discharge product DP1 to be more effectively subjected to the induction effect of the induction electrode 5 compared to the air purifier 1. Therefore, the air purifier 1A suppresses the diffusion of the discharge product DP1 more effectively than the air purifier 1, resulting in a higher concentration. As a result, the air purifier 1A can transport the discharge product DP1 at a higher concentration compared to the air purifier 1 to the treatment target, thereby enhancing its sterilization and virus inactivation effects.

### Embodiment 3

An air purifier 1B according to Embodiment 3 differs from the air purifier 1 in Embodiment 1 and the air purifier 1A in Embodiment 2 in that it additionally includes a second discharge electrode and has a different shape for the induction electrode 5 Hereafter, the explanation will focus on the points where Embodiment 3 differs from Embodiment 1. Configurations not described in Embodiment 3 are the same as those in Embodiment 1.

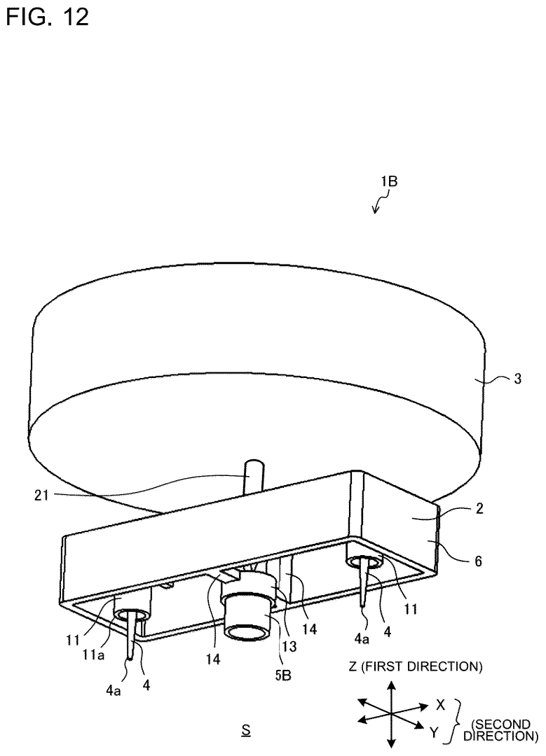

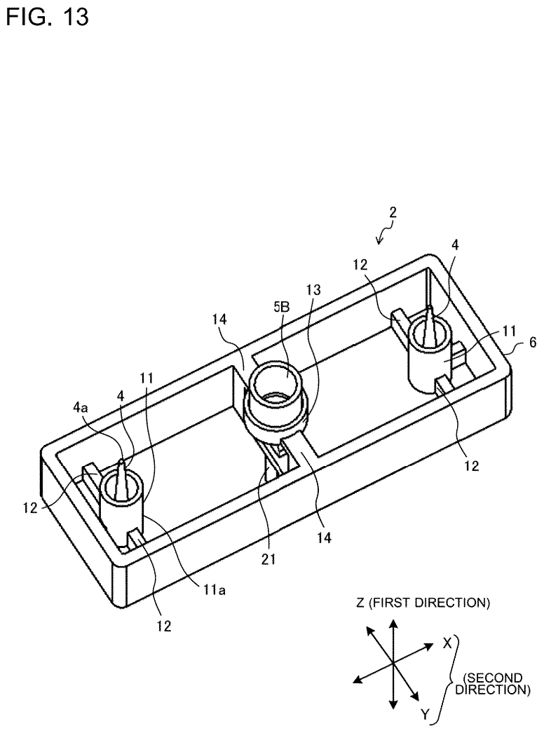

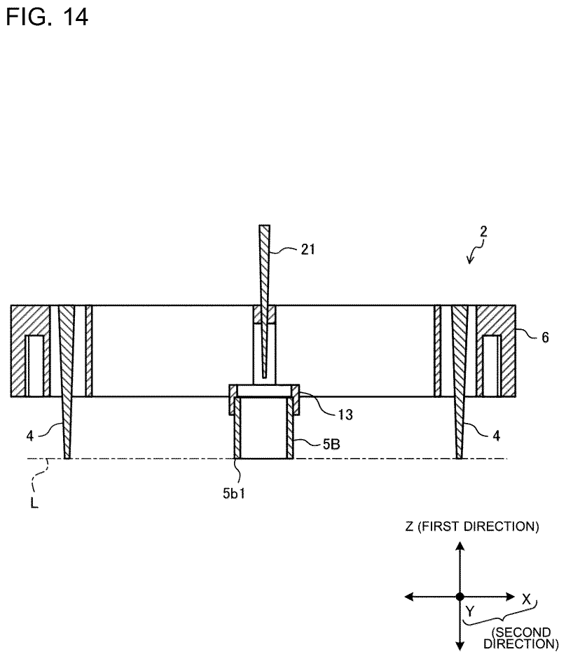

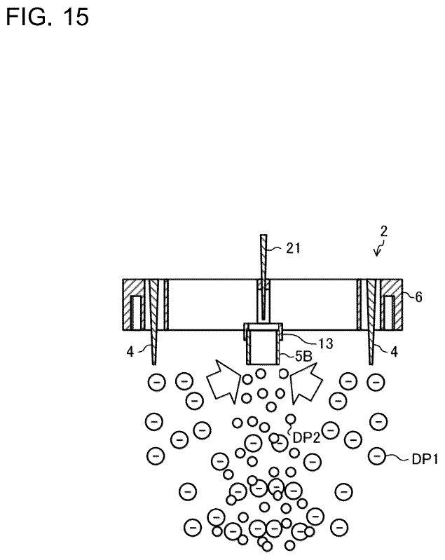

FIG. 12 is a schematic perspective view of the air purifier 1B according to Embodiment 3. FIG. 13 is a schematic perspective view of the generating unit 2 of the air purifier 1B according to Embodiment 3, as viewed from below. FIG. 14 is a schematic cross-section of the generating unit 2 of the air purifier 1B according to Embodiment 3. FIG. 15 is a schematic diagram illustrating the behaviors of the discharge product DP1 and the discharge product DP2 of the air purifier 1B according to Embodiment 3.

The air purifier 1B includes a second discharge electrode 21 in addition to the configuration of the air purifier 1. The second discharge electrode 21 is either enclosed in an electrically insulating resin part (not shown) and fixed to the second retaining unit 13 or fixed to the second retaining unit 13 by screws or adhesive. Furthermore, the air purifier 1B has an induction electrode 5B with a shape differing from the induction electrode 5 in Embodiment 1, being formed in a cylindrical shape. The induction electrode 5B is designed to have the cylindrical shape to guide the discharge product DP2, generated between it and the second discharge electrode 21, toward the treatment target W. As shown in FIG. 14, the induction electrode 5B is positioned such that its tip-end surface 5b1 overlaps the virtual line L. However, it may also be arranged on the treatment target W side from the virtual line L in the first direction.

The second discharge electrode 21, like the first discharge electrode 4, extends in the first direction. Specifically, the second discharge electrode 21 is formed as a conical needle-shaped electrode, tapering from its base end toward its tip end. The second discharge electrode 21 is positioned inside the induction electrode 5B when viewed in the first direction. The second discharge electrode 21 is positioned inside the induction electrode 5B when viewed in the first direction. The second discharge electrode is positioned at the center of the induction electrode 5B when viewed in the first direction. In the first direction, the second discharge electrode 21 is located on the side opposite to the treatment target W across the induction electrode 5B. The tip of the second discharge electrode 21 is arranged to face the induction electrode 5B in the first direction. The second discharge electrode 21 is spaced apart from the induction electrode 5B in the first direction. However, it is not strictly necessary for the second discharge electrode 21 to be spaced apart; from the induction electrode 5B; it may also be positioned to overlap with the induction electrode 5B in the first direction. Specifically, the bottom end of the second discharge electrode 21 may be inserted into the internal space of the induction electrode 5B.

As shown in FIG. 15, the second discharge electrode 21 generates, with the induction electrode 5B, a discharge therebetween when a high voltage obtained from a high-voltage converter (not shown) is applied. This results in the production of a discharge product DP2, which is different from the discharge product DP1. The discharge distance between the second discharge electrode 21 and the induction electrode 5B is arranged to be shorter than the discharge distance between the first discharge electrode 4 and the induction electrode 5B. By having a shorter discharge distance between the second discharge electrode 21 and the induction electrode 5B compared to the discharge distance between the first discharge electrode 4 and the induction electrode 5B, the second discharge electrode 21 generates the discharge product DP2, which differs from the discharge product DP1.

The second discharge electrode 21 is described as being composed of a needle-shaped electrode; however, it is not limited to this shape. To concentrate the electric field, a tapered tip end shape is desirable for the second discharge electrode 21, but it is not restricted to this shape and may also be columnar. Additionally, the second discharge electrode 21 can take the form of an electrode made of equal thickness fine wires or a brush-shaped electrode composed of a bundle of multiple thin wires. The material of the second discharge electrode 21 is metal. However, the material is not limited to metal and may also be formed from other conductive materials, such as carbon fibers or other conductive substances.

The discharge product DP2 generated by the second discharge electrode 21 differs from the discharge product DP1 generated by the first discharge electrode 4, as described above. Specifically, for example, the discharge product DP1 is an ion, whereas the discharge product DP2 is ozone. Because the discharge distance between the second discharge electrode 21 and the induction electrode 5B is shorter than the discharge distance between the first discharge electrode 4 and the induction electrode 5B, electrons emitted from the second discharge electrode 21 are more easily accelerated between the second discharge electrode 21 and the induction electrode 5B, resulting in a higher energy state. Thus, the electrons in the region between the second discharge electrode 21 and the induction electrode 5B may include electrons with energy levels exceeding the dissociation energy of oxygen molecules in the air, which is 5.12 eV. High-energy electrons collide with oxygen molecules in the air, leading to dissociation. The dissociated oxygen atoms and oxygen molecules then undergo three-body collisions, resulting in the formation of ozone. It should be noted that the differences between discharge products DP1 and DP2 are not limited to ions and ozone. For instance, they may also differ in the ratio or concentration of reactive species.

As described above, the air purifier 1B generates the discharge product DP2 which differs from the discharge product DP1, from the second discharge electrode 21, in addition to the discharge product DP1 generated by the first discharge electrode 4. Therefore, the air purifier 1B can process the treatment target W using both the discharge product DP1 and the discharge product DP2, thereby enhancing its bactericidal and virus inactivation effects.

Since the induction electrode 5B is cylindrical, at least a portion of the discharge product DP2 generated by the second discharge electrode 21 passes through the internal space of the induction electrode 5B and is directed toward the treatment target W. In this way, the induction electrode 5B acts as a guiding pathway for the discharge product DP2 into the space S, thereby suppressing its diffusing of the discharge product DP2 and increasing the concentration.

[Effects of Air Purifier 1b]

The air purifier 1B achieves the same effects as those of Embodiment 1, along with the following additional benefits. The air purifier 1B includes the second discharge electrode 21 that generates the discharge product DP2, which differs from the discharge product DP1 generated by the first discharge electrode 4. As a result, the air purifier 1B can process the treatment target W using both the discharge product DP1 and the discharge product DP2, thereby enhancing its sterilization and virus-inactivation effects.

Furthermore, the air purifier 1B has the discharge distance between the second discharge electrode 21 and the induction electrode 5B that is shorter than the discharge distance between the first discharge electrode 4 and the induction electrode 5B. As a result, the second discharge electrode 21 generates the discharge product DP2, which differs from the discharge product DP1. The air purifier 1B has the cylindrical induction electrode 5B, with the second discharge electrode 21 positioned inside the cylindrical induction electrode 5B when viewed in the first direction. This configuration allows at least a portion of the discharge product DP2 to pass through the internal space of the cylindrical induction electrode 5B, guiding it toward the treatment target W and suppressing the diffusion of the discharge product DP2. Consequently, the air purifier 1B can increase the concentration of not only the discharge product DP1 but also the discharge product DP2. As a result, the air purifier 1B can transport both discharge product DP1 and discharge product DP2 at high concentrations to the treatment target W, thereby enhancing its sterilization and virus-inactivation effects.

### Embodiment 4

An air purifier 1C according to Embodiment 4 differs from the air purifier 1B of Embodiment 3 in that it further includes a shielding electrode 31. Hereafter, the explanation will focus on the differences between Embodiment 4 and Embodiment 3, and any configurations not explained for Embodiment 4 are the same as those in Embodiment 3.

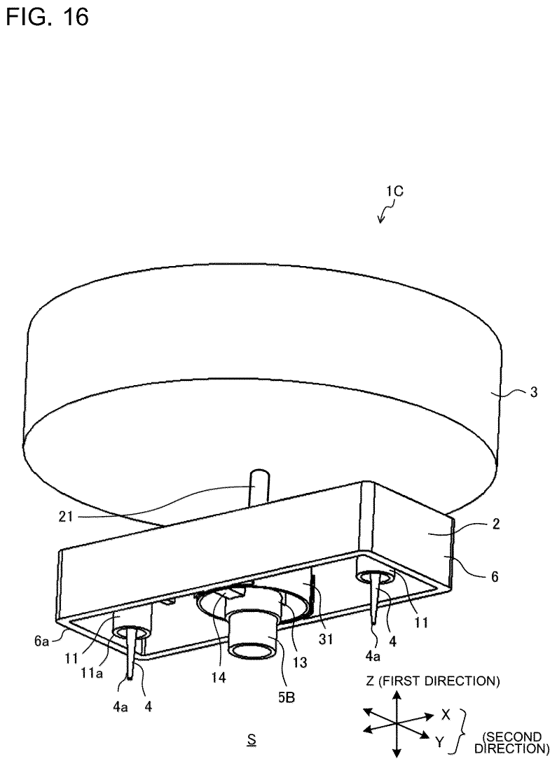

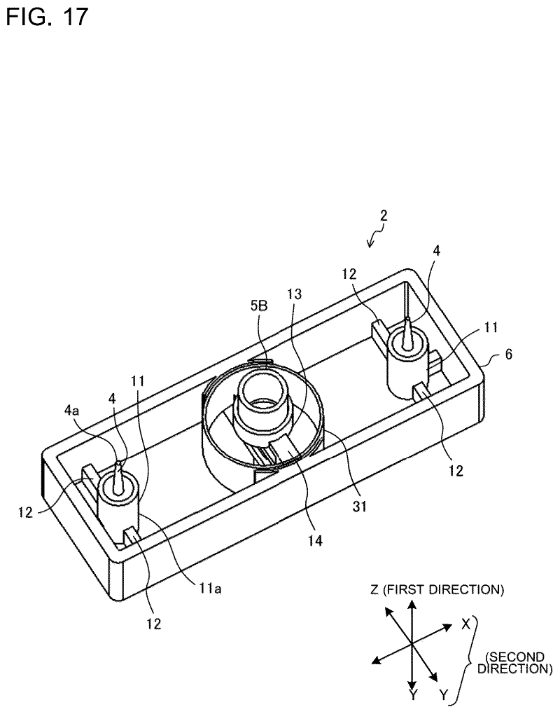

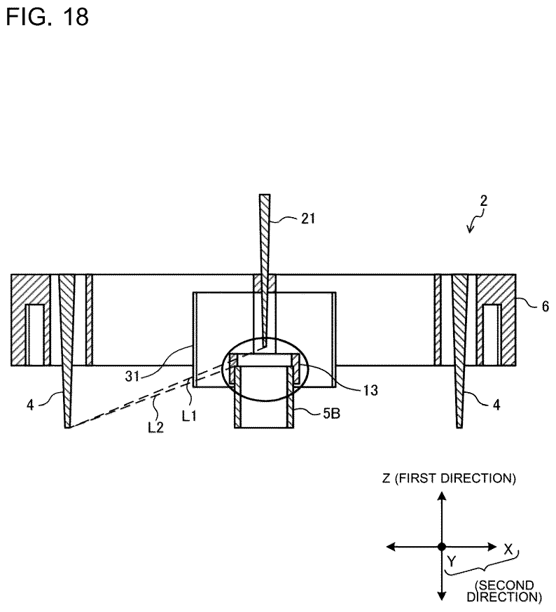

FIG. 16 is a schematic perspective view of the air purifier 1C according to Embodiment 4. FIG. 17 is a schematic perspective view of the generating unit 2 of the air purifier 1C according to Embodiment 4, as viewed from below. FIG. 18 is a schematic cross-section of the generating unit 2 of the air purifier 1B according to Embodiment 4.

The air purifier 1C, in addition to the configuration of the air purifier 1B, includes the shielding electrode 31 to prevent the static charge of the retaining member 6 caused by the discharge product DP1. The shielding electrode 31 is cylindrically shaped and surrounds the induction electrode 5B and the second discharge electrode 21. The shielding electrode 31 surrounds the area around the end of the induction electrode 5B on the side of the second discharge electrode 21, and the end of the second discharge electrode 21 on the side of the induction electrode 5B. The shielding electrode 31 is arranged to intersect both line L1, connecting the tip of the first discharge electrode 4 and the tip of the second discharge electrode 21, and line L2, connecting the tip of the first discharge electrode 4 and the upper end of the induction electrode 5B.

The shielding electrode 31 prevents a charge-up phenomenon where the discharge product DP1 generated by the first discharge electrode 4 adheres to the surface of the retaining member 6 near the second discharge electrode 21, causing the surface of the retaining member 6 to become statically charged. Additionally, the shielding electrode 31 can prevent not only charge-up phenomena of those caused by DP1 but also those caused by DP2 when DP2 has polarity and adheres to the surface of the retaining member 6 near the second discharge electrode 21.

The shielding electrode 31 is grounded, allowing charges to be released from the system through grounding. The shielding electrode 31 prevents the charge-up phenomenon by enabling discharge product DP1 to make contact and release its charge externally. Therefore, the material of the shielding electrode 31 is metal. However, the material is not limited to metal; for example, it can be made of other conductive materials such as carbon fibers with conductivity. The shielding electrode 31, as shown in FIG. 18 within the encircled area, shields the portion where the induction electrode 5B and the second discharge electrode 21 face each other from the electric field formed by the first discharge electrode 4, thereby preventing the charge-up phenomenon.

If charge-up cannot be prevented, it becomes impossible to maintain the electric field of 25 [kV/cm] required for discharge. Consequently, discharge products DP1 and DP2 will no longer be generated stably, resulting in a decrease in their concentrations. In contrast, the air purifier 1C can prevent charge-up, enabling the stable generation of discharge products DP1 and DP2 and allowing their concentrations to be increased.

### [Effects of Air Purifier 1C]

The air purifier 1C achieves the same effects as the air purifier 1B, along with the following additional benefits. The air purifier 1C includes the shielding electrode 31 that surrounds the induction electrode 5B and the second discharge electrode 21. As a result, the air purifier 1C prevents charge-up, ensuring the stable generation of discharge products DP1 and DP2 and increasing their concentrations. Consequently, the air purifier 1C can deliver discharge products DP1 and DP2 to the treatment target W at higher concentrations compared to the air purifier 1B, thereby enhancing sterilization and virus inactivation effects.

### Embodiment 5

Embodiment 5 relates to an air-conditioning apparatus including any of the air purifiers described in embodiments 1 through 4. Hereafter, an example is provided where the air-conditioning apparatus includes the air purifier 1 of Embodiment 1.

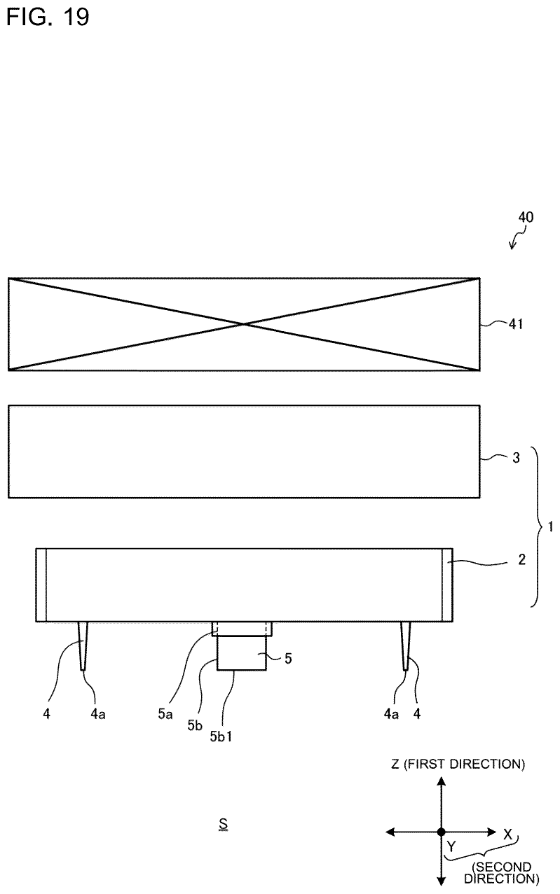

FIG. 19 illustrates a schematic diagram of an example of the air-conditioning apparatus 40 described in Embodiment 5. The air-conditioning apparatus 40 includes an air purifier 1 and a heat exchanger 41, which performs heat exchange between the refrigerant flowing inside the heat exchanger 41 and the air surrounding the heat exchanger 41. The air-conditioning apparatus 40 supplies air transmitted by the air blowing unit 3, which passes through the heat exchanger 41. After passing through the heat exchanger 41, the conditioned air delivers the discharge product DP1 into the space S. In FIG. 19, the air purifier 1 is shown equipped with an air blowing unit 3; however, any configuration is applicable as far as the air blowing unit 3 is integrated within the air-conditioning apparatus 40 itself.

The air-conditioning apparatus 40 with the above-described configuration incorporates the air purifier 1, enabling the system to transport the discharge product DP1 generated by the air purifier 1 toward the treatment target W in a highly concentrated state. This configuration delivers enhanced sterilization and virus inactivation effects.

The configurations described in the above embodiments represent examples of the content of the present disclosure, which can be combined with other known techniques, and parts of the configurations may be omitted or modified as long as such changes do not deviate from the gist of the present disclosure.

### REFERENCE SIGNS LIST

1: air purifier, 1A: air purifier, 1B: air purifier, 1C: air purifier, 2: generating unit, 3: air blowing unit, 4: first discharge electrode, 4a: discharge point, 5: induction electrode, 5B: induction electrode, 5a: base end, 5b: tip end, 5b1: tip-end surface, 6: retaining member, 11: first retaining unit, 11a: bottom surface, 12: fixing leg, 13: second retaining unit, 14: fixing leg, 15: frame portion, 15a: bottom surface, 21: second discharge electrode, 25: electric field, 31: shielding electrode, 40: air-conditioning apparatus, 41: heat exchanger, D1: discharge product, DP1: discharge product, DP2: discharge product, DPg: discharge product group, DPs1: discharge product, DPs2: discharge product, EF: electric field, L: virtual line, L1: line connecting the tip end of the first discharge electrode and the tip of the second discharge electrode, L2: line connecting the tip of the first discharge electrode and the upper end of the induction electrode, O: central axis, S: space, W treatment target.

1. An air purifier including a plurality of first discharge electrodes and being configured to supply a discharge product generated by the plurality of first discharge electrodes toward a treatment target within a space to purify the air in the space,
the plurality of first discharge electrodes being formed to extend in a first direction, and being electrodes of a same polarity arranged spaced apart in a second direction orthogonal to the first direction,
the air purifier comprising
an induction electrode arranged at a central part of the plurality of first discharge electrodes as viewed in the first direction, spaced apart in a first direction from the plurality of the first discharge electrode, to extend in the first direction, and configured to form, with the plurality of first discharge electrodes, an electric field between the induction electrode and the plurality of first discharge electrodes to attract the discharge product generated from the plurality of first discharge electrodes, and
an air-blowing unit configured to form a flow of air in the first direction,
wherein each of the first discharge electrodes has, on one side of both ends in the first direction, a discharge point that comprises a needle-shaped tip and at which discharge occurs, and
the induction electrode includes
a base end and a tip end located on a side opposite to a side where the base end is present, the side being the one side in the first direction, the tip end having a tip-end surface being configured to overlap, in the first direction, a virtual line connecting the discharge points of the plurality of first discharge electrodes, or positioned on a side, where the treatment target is present, from the virtual line.

2. The air purifier of claim 1, further comprising:
a retaining member having an electrical insulation property, and configured to retain the plurality of first discharge electrodes and the induction electrode,
wherein each of the plurality of first discharge electrodes is retained by the retaining member at an end portion opposite to the discharge point.

3. (canceled)

4. The air purifier of claim 1, further comprising:
a second discharge electrode configured to generate a discharge product different from the discharge product generated by the plurality of first discharge electrodes.

5. The air purifier of claim 4, wherein:
the induction electrode has a cylindrical shape,
the second discharge electrode is arranged inside the induction electrode as viewed in the first direction,
a discharge distance between the second discharge electrode and the induction electrode is shorter than a discharge distance between the plurality of first discharge electrodes and the induction electrode.

6. The air purifier of claim 2, further comprising:
a second discharge electrode configured to generate a discharge product different from the discharge product generated by the plurality of first discharge electrodes; and
a shielding electrode having a cylindrical shape and surrounding the induction electrode and the second discharge electrode, and preventing the electrification of the retaining member caused by the discharge product.

7. An air-conditioning apparatus comprising:
the air purifier of claim 1; and
a heat exchanger configured to cause refrigerant flowing inside the heat exchanger and air present around the heat exchanger to exchange heat with each other,
wherein the air-conditioning apparatus is configured such that the air supplied by the air blowing unit arranged upstream of the plurality of first discharge electrodes passes through the heat exchanger, is air-conditioned by passing through the heat exchanger, and supplies the discharge product into the space.
